(19) United States
(12) Patent Application Publication
(10) Pub. No.: US 2007/0185162 A1
(43) Pub. Date: Aug. 9, 2007

(54) Substituted diketopiperazines as oxytocin receptor antagonists

(75) Inventors: Alan David Borthwick, Stevenage Hertfordshire (GB); Deidre Mary Bernadette Hickey, Stevenage Hertfordshire (GB); John Liddle, Stevenage Hertfordshire (GB); Andrew McMurtrie Mason, Stevenage Hertfordshire (GB); Derek Roland Pollard, Stevenage Hertfordshire (GB); Steven Sollis, Stevenage Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Koninklijke Philips Electronics, N.V., Einndhoveen (NL)

(21) Appl. No.: 11/630,100

(22) PCT Filed: Jun. 21, 2005
(86) PCT No.: PCT/GB05/02448

§ 371 Date: Dec. 19, 2006

(30) Foreign Application Priority Data
Jun. 23, 2004 (GB) 0414100.8

(51) Int. Cl. A61K 31/445; C07D 241/04

(52) U.S. Cl. 514/315; 544/384

(57) ABSTRACT

Compounds of formula (I) 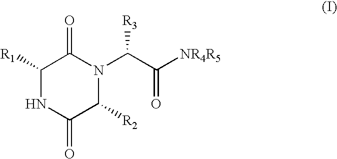
wherein R₁ is 2-indanyl, R₂ is 1-ethylpropyl, R₃ is a heterocyclic group optionally substituted by one or more C₁₋₆ alkyl groups, R₄ represents methyl and R₅ represents hydrogen or methyl and pharmaceutically acceptable derivatives thereof are described, as are processes for their preparation, pharmaceutical compositions containing them and their use in medicine, particularly their use as oxytocin antagonists.

This invention relates to novel diketopiperazine derivatives having a potent and selective antagonist action at the oxytocin receptor, to processes for their preparation, pharmaceutical compositions containing them and to their use in medicine.

The hormone oxytocin is a potent contractor of the uterus and is used for the induction or augmentation of labour. Also the density of uterine oxytocin receptors increases significantly by >100 fold during pregnancy and peaks in labour (pre-term and term).

Pre-term births/labour (between 24 and 37 weeks) causes about 60% of infant mortality/morbidity and thus a compound which inhibits the uterine actions of oxytocin e.g. oxytocin antagonists, should be useful for the prevention or control of pre-term labour.

International patent application WO 99/47549 describes diketopiperazine derivatives including 3-benzyl-2,5-diketopiperazine derivatives as inhibitors of fructose 1,6-bisphosphate (FBPase).

International patent application WO 03/053443 describes a class of diketopiperazine derivatives which exhibit a particularly useful level of activity as selective antagonists at the oxytocin receptor. A preferred class of compounds described therein is represented by the formula (A):

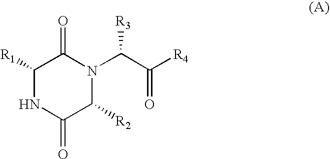

Such compounds include those wherein inter alia R₁ is 2-indanyl, R₂ is C₃₋₄alkyl, R₃ is a 5- or 6-membered heteroaryl group linked to the rest of the molecule via a carbon atom in the ring, R₄ represents the group NR₅R₆ wherein R₅ and R₆ each represent alkyl e.g. methyl, or R₅ and R₆ together with the nitrogen atom to which they are attached form a 3- to 7-membered saturated heterocyclic ring which heterocycle may contain an additional heteroatom selected from oxygen.

International patent application WO 2005/000840 describes diketopiperazine derivatives of formula (B) 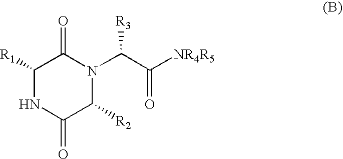
wherein R₁ is 2-indanyl, R₂ is 1-methylpropyl, R₃ is 2-methyl-1,3-oxazol-4-yl and R₄ and R₅ together with the nitrogen atom to which they are attached represent morpholino.

We have now found a novel group of selective oxytocin receptor antagonists which exhibit a particularly advantageous pharmacokinetic profile.

The present invention thus provides at least one chemical entity selected from compounds of formula (I) 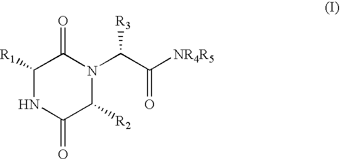
wherein R₁ is 2-indanyl, R₂ is 1-ethylpropyl, R₃ is a heterocyclic group optionally substituted by one or more C₁₋₆ alkyl groups, R₄ represents methyl and R₅ represents hydrogen or methyl and pharmaceutically acceptable derivatives thereof.

The present invention also provides at least one chemical entity selected from compounds of formula (IA) 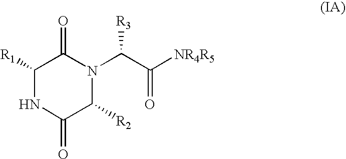
wherein R₁ is a 2-indanyl, R₂ is 1-ethylpropyl, R₃ is 6-methyl-3-pyridinyl, R₄ represents methyl and R₅ represents hydrogen or methyl and pharmaceutically acceptable derivatives thereof.

It will be appreciated that the compounds of formula (I) and formula (IA) possess the absolute stereochemistry depicted at the asymmetric carbon atoms bearing groups R₁, R₂ and R₃, ie the stereochemistry at these positions is always (R). Nevertheless, it should also be appreciated that although such compounds are substantially free of the (S)-epimer at each of R₁, R₂ and R₃, each epimer may be present in small amounts, for example 1% or less of the (S)-epimer may be present.

In one embodiment of the invention, R₃ is indazolyl, pyridinyl or oxazolyl, any of which may be optionally substituted by one or more C₁₋₆ alkyl groups. In another embodiment R₃ is indazolyl optionally substituted by one or more C₁₋₆ alkyl groups. In yet another embodiment R₃ is pyridinyl optionally substituted by one or more C₁₋₆ alkyl groups. In yet another embodiment R₃ is 6-methyl-3-pyridinyl. In a further embodiment R₃ is 2,6-dimethyl-3-pyridinyl. In a further embodiment R₃ is oxazolyl optionally substituted by one or more C₁₋₆ alkyl groups. In another embodiment R₃ is 2-methyl-4-oxazolyl.

In one embodiment of the invention is the compounds the preparation of which is specifically described in examples 1 to 9. Another embodiment of the invention is the compounds the preparation of which is specifically described in examples 1, 3, 6 and 9. A further embodiment of the invention is the compounds the preparation of which is specifically described in examples 1 and 9. In another embodiment of the invention is the compound the preparation of which is specifically described in example 1. In yet another embodiment of the invention is the compound the preparation of which is specifically described in example 9.

In one aspect, chemical entities useful in the present invention may be at least one chemical entity selected from:
(2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide;
(2R)-2-[(3R,6R)-3-(2,3-Dihydro-1H-inden-2-yl)-6-(1-ethylpropyl)-2,5-dioxo-1-piperazinyl]-N,N-dimethyl-2-(6-methyl-3-pyridinyl)ethanamide;
(2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N-methylethanamide;
(2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N,N-dimethylethanamide;
(3R,6R)-3-(2,3-Dihydro-1H-inden-2-yl)-1-[(1R)-1-(2,6-dimethyl-3-pyridinyl)-2-(4-morpholinyl)-2-oxoethyl]-6-(1-ethylpropyl)-2,5-piperazinedione;
(2R)-2-[(3R,6R)-3-(2,3-Dihydro-1H-inden-2-yl)-6-(1-ethylpropyl)-2,5-dioxo-1-piperazinyl]-N-methyl-2-(1-methyl-1H-indazol-5-yl)acetamide;
(2R)-2-[(3R,6R)-3-(2,3-Dihydro-1H-inden-2-yl)-6-(1-ethylpropyl)-2,5-dioxo-1-piperazinyl]-N,N-dimethyl-2-(1-methyl-1H-indazol-5-yl)ethanamide;
(3R,6R)-3-(2,3-Dihydro-1H-inden-2-yl)-6-(1-ethylpropyl)-1-[(1R)-1-(1-methyl-1H-indazol-5-yl)-2-(4-morpholinyl)-2-oxoethyl]-2,5-piperazinedione;
(3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione;
   and pharmaceutically acceptable derivatives thereof.

As used herein, the term “pharmaceutically acceptable” means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

As used herein, the term “pharmaceutically acceptable derivative”, means any pharmaceutically acceptable salt, solvate, or prodrug e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. In one aspect, pharmaceutically acceptable derivatives are salts, solvates, esters, carbamates and phosphate esters. In another aspect, pharmaceutically acceptable derivatives are salts, solvates and esters. In one aspect, pharmaceutically acceptable derivatives are physiologically acceptable salts. In a further aspect, pharmaceutically acceptable derivatives are solvates and esters. In another aspect, pharmaceutically acceptable derivatives are solvates.

Suitable physiologically acceptable salts of compounds of the present invention include acid addition salts formed with physiologically acceptable inorganic acids or organic acids. Examples of such acids include hydrochloric acid, hydrobromic acid, nitric acid, phosphoric acid, sulphuric acid, sulphonic acids e.g. methanesulphonic, ethanesulphonic, benzenesulphonic and p-toluenesulphonic, citric acid, tartaric acid, lactic acid, pyruvic acid, acetic acid, succinic acid, fumaric acid and maleic acid.

The present invention also relates to solvates of the compounds of formula (I) or formula (IA), for example hydrates, or solvates with pharmaceutically acceptable solvents including, but not limited to, alcohols, for example ethanol, iso-propanol, acetone, ethers, esters, e.g. ethyl acetate.

As used herein, the term “heterocyclic group” means a 5- or 6-membered monocyclic or fused bicyclic heterocyclic group containing at least one heteroatom selected from oxygen, sulphur or nitrogen. Examples of such 5-membered heterocyclic groups include but are not limited to furanyl, thienyl, pyrrolyl, oxazolyl, isoxazolyl, thiazolyl, imidazolyl, pyrazolyl, oxadiazolyl, thiadiazolyl, triazolyl, or tetrazolyl. Examples of such 6-membered heterocyclic groups include but are not limited to pyridinyl, pyrimidinyl and triazinyl. The fused bicyclic heterocyclic group may also conveniently be a 6,5- or 6,6-fused ring system wherein the heterocycle may be partially saturated, or together with the benzene ring to which it is fused to form a heteroaryl group. Examples of fused 6,6-heterocyclic groups include but are not limited to quinolinyl, isoquinolinyl, phthalazinyl, cinnolinyl, quinazolinyl, quinoxalinyl, 1,2,3-benzotriazinyl or 1,2,4-benzotriazinyl. Examples of fused 6,5-heterocyclic groups include but are not limited to benzofuranyl, benzothienyl, indolyl, benzo-oxadiazolyl, benzothiadiazolyl, benzo-oxazolyl, benzothiazolyl, benzoisothiazolyl, benzoisoxazolyl, benzimidazolyl, indazolyl or benzotriazolyl.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with tocolytics or prophylactic medicines. These include, but are not limited to, beta-agonists such as terbutaline or ritodrine, calcium channel blockers, e.g. nifedepine, non-steroidal anti-inflammatory drugs, such as indomethacin, salts of magnesium, such as magnesium sulphate, other oxytocin antagonists, such as atosiban, and progesterone agonists and formulations. In addition the compounds of the present invention may be used in combination with antenatal steroids including betamethasone and dexamethasone, prenatal vitamins especially folate supplements, antibiotics, including but not limited to ampicillin, amoxicillin/clavulanate, metronidazole, clindamycin, and anxiolytics.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compounds of formula (I) and formula (IA) have a high affinity for the oxytocin receptors on the uterus of rats and humans and this may be determined using conventional procedure. For example the affinity for the oxytocin receptors on the rat uterus may be determined by the procedure of Pettibone et al, Drug Development Research 30. 129-142(1993). The compounds of the invention also exhibit high affinity at the human recombinant oxytocin receptor in CHO cells and this may be conveniently demonstrated using the procedure described by Wyatt et al. Bioorganic & Medicinal Chemistry Letters, 2001 (11) p 1301-1305.

The compounds of the invention exhibit an advantageous pharmacokinetic profile including good potency at the oxytocin receptor coupled with good aqueous solubility.

The compounds of the invention are therefore useful in the treatment or prevention of diseases and/or conditions mediated through the action of oxytocin. Examples of such diseases and/or conditions include pre-term labour, dysmenorrhea, endometriosis and benign prostatic hyperplasia.

The compounds may also be useful to delay labour prior to elective cesarean section or transfer of the patient to a tertiary care centre, treatment of sexual dysfunction (male and female), particularly premature ejaculation, obesity, eating disorders, congestive heart failure, arterial hypertension, liver cirrhosis, nephritic or ocular hypertension, obsessive-compulsive disorder and neuropsychiatric disorders. The compounds of the invention may also be useful for improving fertility rates in animals, e.g. farm animals.

The invention therefore provides for at least one chemical entity selected from compounds of formula (I) or formula (IA), and pharmaceutically acceptable derivatives thereof for use in therapy, particularly for use in human or veterinary therapy, and in particular for use as a medicine for antagonising the effects of oxytocin upon the oxytocin receptor.

The invention also provides for the use of at least one chemical entity selected from compounds of formula (I) or formula (IA), and pharmaceutically acceptable derivatives thereof for the manufacture of a medicament for antagonising the effects of oxytocin on the oxytocin receptor.

According to a further aspect, the invention also provides for a method for antagonising the effects of oxytocin upon the oxytocin receptor, comprising administering to a patient in need thereof an antagonistic amount of at least one chemical entity selected from compounds of formula (I) and formula (IA) and pharmaceutically acceptable derivatives thereof.

It will be appreciated by those skilled in the art that reference herein to treatment extends to prophylaxis as well as the treatment of established diseases or symptoms.

It will further be appreciated that the amount of a compound of the invention required for use in treatment will vary with the nature of the condition being treated, the route of administration and the age and the condition of the patient and will be ultimately at the discretion of the attendant physician. In general however doses employed for adult human treatment will typically be in the range of 2 to 1000 mg per day, dependent upon the route of administration.

Thus for parenteral administration a daily dose will typically be in the range 2 to 50 mg, preferably 5 to 25 mg per day. For oral administration a daily dose will typically be within the range 10 to 1000 mg, e.g. 50 to 500 mg per day.

The desired dose may conveniently be presented in a single dose or as divided doses administered at appropriate intervals, for example as two, three, four or more sub-doses per day.

While it is possible that, for use in therapy, a compound of the invention may be administered as the raw chemical, it is preferable to present the active ingredient as a pharmaceutical formulation.

The invention thus further provides a pharmaceutical formulation comprising at least one chemical entity selected from compounds of formula (I) and (IA), and pharmaceutically acceptable derivatives thereof, together with one or more pharmaceutically acceptable carriers thereof and, optionally, other therapeutic and/or prophylactic ingredients. The carrier(s) must be ‘acceptable’ in the sense of being compatible with the other ingredients of the formulation and not deleterious to the recipient thereof.

The compositions of the invention include those in a form especially formulated for oral, buccal, parenteral, inhalation or insufflation, implant, vaginal or rectal administration.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcrystalline cellulose, maize-starch, calcium phosphate or sorbitol; lubricants, for example, magnesium stearate, stearic acid, talc, polyethylene glycol or silica; disintegrants, for example, potato starch or sodium starch glycollate, or wetting agents such as sodium lauryl sulphate. The tablets may be coated according to methods well known in the art. Oral liquid preparations may be in the form of, for example, aqueous or oily suspensions, solutions emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxyethylcellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; solubilizers such as surfactants for example polysorbates or other agents such as cyclodextrins; and preservatives, for example, methyl or propyl p-hydroxybenzoates or ascorbic acid. The compositions may also be formulated as suppositories, e.g. containing conventional suppository bases such as cocoa butter or other glycerides.

For buccal administration the composition may take the form of tablets or lozenges formulated in conventional manner.

The composition according to the invention may be formulated for parenteral administration by injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

The compositions according to the invention may contain between 0.1-99% of the active ingredient, conveniently from 1-50% for tablets and capsules and 3-50% for liquid preparations.

The advantageous pharmacokinetic profile of the compounds of the invention is readily demonstrated using conventional procedures for measuring the pharmacokinetic properties of biologically active compounds.

The compounds of the invention and pharmaceutically acceptable derivatives thereof may be prepared by the processes described hereinafter, said processes constituting a further aspect of the invention. In the following description, the groups are as defined above for compounds of the invention unless otherwise stated.

Compounds of formula (I) and formula (IA) may be prepared by reaction of the carboxylic acid (II), wherein R₁, R₂ and R₃ have the meanings defined in formula (I) and formula (IA), and the chirality at R₃ is either (R) or (S), or a mixture thereof, 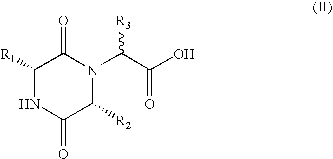
or an activated derivative thereof with the amine HNR₄R₅ wherein R₄ and R₅ have the meanings defined in formula (I) and formula (IA) under standard conditions for preparing amides from a carboxylic acid or a mixed anhydride thereof and an amine HNR₄R₅.

It will be appreciated that the mixture of diastereomers of compounds of formula (i) and formula (IA) obtained from the above reaction may be separated using standard resolution techniques well known in the art, for example column chromatography.

Thus the amide of formula (I) or formula (IA) may be prepared by treating the carboxylic acid of formula (II) with an activating agent such as BOP (benzotriazol-1-yloxy-tris(dimethylamino)phosphonium hexafluorophosphate), TBTU (2-(1H-benzotriazol-1-yl)-1,1,3,3-tetramethyluronium tetrafluoroborate), BOP-CI (bis(2-oxo-3-oxazolidinyl)phosphinic chloride), oxalyl chloride or 1,1′-carbonyldiimidazole in an aprotic solvent such as dichloromethane optionally in the presence of a tertiary amine such as triethylamine and subsequent reaction of the product thus formed, ie the activated derivative of the compound of formula (II), with the amine HNR₄R₅.

Alternatively the amide of formula (I) or formula (IA) may be prepared by reacting a mixed anhydride derived from the carboxylic acid (II) with the amine HNR₄R₅ in an aprotic solvent such as tetrahydrofuran. Conveniently the reaction is carried out at low temperatures, for example 25° C. to −90° C., more conveniently at approximately −78° C.

The mixed anhydride is conveniently prepared by reacting the carboxylic acid (II) with a suitable acid chloride e.g. pivalolyl chloride in an aprotic solvent such as ethyl acetate in the presence of a tertiary organic base such as a trialkylamine e.g. triethylamine and at low temperatures, for example 25° C. to −90° C., more conveniently at approximately −78° C.

Compounds of formula (I) or formula (IA) may also be prepared by reacting a compound of formula (III) 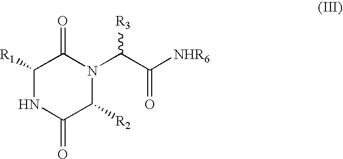
wherein R₁, R₂ and R₃ have the meanings defined in formula (I) or formula (IA) and R₆ is 2-hydroxyphenyl, with 1,1′-carbonyldiimidazole or 1,1′-thiocarbonyldiimidazole in a suitable solvent such as dichloromethane and subsequent reaction of the products this formed with the amine HNR₄R₅.

Compounds of formula (II) may be prepared from a compound of formula (III) wherein R₆ is 2-hydroxyphenyl by reaction with 1,1′-carbonyldiimidazole or 1,1-thiocarbonyldiimidazole in a suitable solvent such as dichloromethane and subsequent reaction of the product thus formed with aqueous acetone.

Compounds of formula (III) wherein R₆ is 2-hydroxyphenyl may be prepared from the corresponding compounds of formula (III) wherein R₆ is a 2-benzyloxyphenyl group by hydrogenolysis using hydrogen and a palladium catalyst.

Alternatively, compounds of formula (III) wherein R₆ is 2-hydroxphenyl may be prepared from a compound of formula (IV) 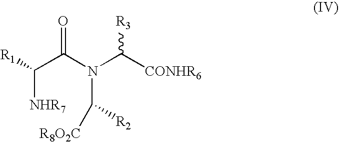
wherein R₁, R₂ and R₃ have the meanings defined in formula (I) and formula (IA), R₆ is 2-benzyloxyphenyl, R₇ is benzyloxycarbonyl and R₈ is C₁₋₆alkyl, by the reaction with hydrogen in the presence of a palladium on charcoal catalyst and acetic acid. This reaction is conveniently carried out in a solvent such as ethanol or trifluoroethanol or mixtures thereof.

Compounds of formula (IV) may be prepared by reacting the amino ester hydrochloride (V), 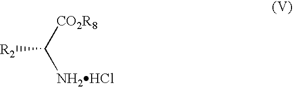
wherein R₂ has the meaning defined in formula (I) and formula (IA) and R₈ is C₁₋₆ alkyl, with the aldehyde R₃CHO (VI) wherein R₃ has the meaning defined in formula (I) or formula (IA), in the presence of triethylamine and in a solvent such as trifluoroethanol and then reacting the resultant product with the compound (VII) 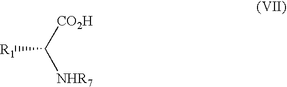
wherein R₁ has the meaning defined in formula (I) and formula (IA) and R₇ is t-butyloxycarbonyl or a benzyloxycarbonyl group and the isocyanide CNR₆ (VIII) wherein R₆ is a 2-benzyloxyphenyl group, in a solvent such as trifluoroethanol.

Compounds of formula (III) wherein R₆ is a 2-benzyloxyphenyl group may be prepared from a compound of formula (IV) wherein R₁, R₂ and R₃ have the meanings defined in formula (I), R₆ is 2-benzyloxyphenyl, R₇ is t-butyloxycarbonyl and R₈ is C1-6alkyl, by the reaction with hydrogen chloride in dioxan followed with triethylamine in a solvent such as dichloromethane.

The compound of formula (IV) wherein R₇ is t-butyloxycarbonyl may be prepared by the route described above for the preparation of a compound of formula (IV) wherein R₇ is benzyloxycarbonyl, using a compound of formula (VII) wherein R₇ is t-butyloxycarbonyl.

Alternatively, a compound of formula (V′) may be employed in place of a compound of formula (V) under identical reaction conditions as described above, wherein the compound of formula (V′) has the same structure as described above for (V) but is a racemic mixture of enantiomers instead of the single enantiomer compound (V).

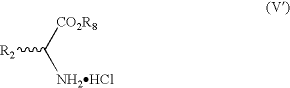

When a compound of formula (V′) is employed, a compounds of formula (IV′) are obtained, which have the same structure as compounds of formula (IV) described above, except that a mixture of epimers at position R₂ is obtained.

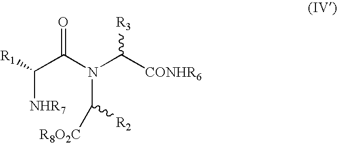

The compounds of formula (IV′) may be subjected to identical reaction conditions as described above for compounds of formula (IV) to obtain compounds of formula (III′), which have the same structure as compounds of formula (III) described above, except that a mixture of epimers at position R₂ is obtained, i.e. a mixture of cis and trans ring isomers at positions R₁ and R₂.

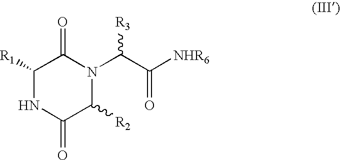

Compounds of formula (III) may be obtained from compounds of formula (III′) by separation, using standard resolution techniques well known in the art, for example column chromatography.

Aminoester hydrochloride (V), wherein R₁ has the meaning defined in formula (I) and formula (IA) and R₈ is C₁₋₆ alkyl, may be prepared from the corresponding N-formyl 3-ethylnorvalinate ester R₂CH(NH₂)CO₂R₈ (X), which may be prepared by separating racemic (NH₂)CO₂R₈ (XI) into its enantiomers by chiral chromatography. In turn (XI) may be prepared from the corresponding 3-ethyl-2-formylamino)-2-pentenoate esters (XII) by reduction using hydrogen and a Pd/C catalyst in a solvent such as acetic acid. Alternatively, chiral reduction of (XII) can give chirally pure (X) directly (JACS, 1995, 117, 9375-9376). The 3-ethyl-2-formylamino)-2-pentenoate esters (XII) can be prepared by literature methods from commercially available starting materials (Chem Ber, 1975, 108, 3079). Racemic aminoester (V′) can be prepared directly from N-formyl derivative (XI) without prior chiral resolution.

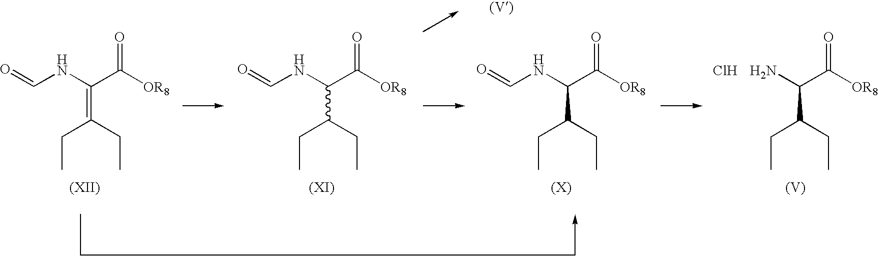

Aldehydes R₃CHO (VI), wherein R₃ has the meaning defined in formula (I) or formula (IA), are either commercially available, or R₃CHO (VI) may be prepared according to standard literature methods, for example, by reduction of the cyano compound R₃CN or esters R₃CO₂Me or R₃CO₂Et, wherein R₃ has the meaning defined in formula (I) or formula (IA), by standard methods such as can be found in J. Org. Chem., 1992, 57 (8) 2235-2244 and J. Org. Chem. 53, 1988, 3513.

The aminoacid derivative (VII) wherein R₁ has the meaning defined in formula (I) and formula (IA) and R₇ is t-butyloxycarbonyl is commercially available; the aminoacid derivative (VII) wherein R₁ has the meaning defined in formula (I) and formula (IA) and R₇ is benzyloxycarbonyl may be prepared from the corresponding commercially available amino acid (R)—R₁CH(NH₂)CO₂H (IX), wherein R₁ has the meaning defined in formula (I) and formula (IA), by treatment with N-(benzyloxycarbonyloxy)succinimde and triethylamine in a solvent such as dioxane in water.

The isocyanide CNR₆ (VIII) may be prepared according to literature methods (Obrecht, Roland; Herrmann, Rudolf; Ugi, Ivar, Synthesis, 1985, 4, 400-402).

Acid addition salts of the compound of formula (I) and formula (IA) may be prepared by conventional means, for example, by treating a solution of the compound in a suitable solvent such as dichloromethane or acetone, with a suitable solution of the appropriate inorganic or organic acid.

The following examples are illustrative, but not limiting of the embodiments of the present invention.

Experimental

Abbreviations

TBTU—2-(1H-benzotriazol-1-yl)-1,1,3,3-tetramethyluronium tetrafluoroborate.

CDI—1,1′-carbonyldiimidazole

LCMS—liquid chromatography-mass spectrometer

Nomenclature

All intermediates and examples were named using ACD Name Pro 6.02 in ISISDraw.

General Purification and Analytical Methods

Analytical HPLC was conducted on a Supelcosil LCABZ+PLUS column (3.3 cm×4.6 mm ID), eluting with 0.1% HCO₂H and 0.01 M ammonium acetate in water (solvent A), and 0.05% HCO₂H and 5% water in acetonitrile (solvent B), using the either elution gradient 1, 0-0.7 minutes 0% B, 0.7-4.2 minutes 0%-100% B, 4.2-5.3 minutes 100% B, 5.3-5.5 minutes 0% B or elution gradient 2, 0-0.7 minutes 0% B, 0.7-4.2 minutes 0%-100% B, 4.2-4.6 minutes 100% B, 4.6-4.8 minutes 0% B at a flow rate of 3 ml/minute. Retention times (Rt) are quoted in minutes. The mass spectra (MS) were recorded on a Waters ZQ 2000 mass spectrometer using electrospray positive [ES+ve to give MH⁺ and M(NH₄)⁺ molecular ions] or electrospray negative [ES-ve to give (M−H)⁻ molecular ion] modes. ¹H NMR spectra were recorded using a Bruker DPX 400 MHz spectrometer using tetramethylsilane as the external standard.

Purification using silica cartridges refers to chromatography carried out using a Combiflash® Companion™ with Redisep® cartridges supplied by Presearch. SPE (solid phase extraction) refers to the use of cartridges sold by International Sorbent Technology Ltd. Preparative HPLC (MDAP) refers to mass-directed auto-preparative HPLC utilizing a HPLCABZ+5 μm column eluting with 0.1% HCO₂H in water and 95% MeCN, 5% water (0.5% HCO₂H) utilising an appropriate gradient elution. A Gilson 202-fraction collector was triggered by a VG Platform Mass Spectrometer on detecting the mass of interest.

Intermediate 1

### Methyl 3-Ethyl-2-(formylamino)-2-pentenoate

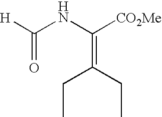

A solution of potassium tert-butoxide (62 g, 550 mmol) in dry tetrahydrofuran (500 ml) was cooled to −78° C. A solution of methylisocyanoacetate (50 ml, 550 mmol) in dry tetrahydrofuran (165 ml) was added keeping the temperature below −65° C. 3-pentanone (58 ml, 550 mmol) was then added as a solution in tetrahydrofuran (165 ml). The reaction was allowed to warm to room temperature and stirred for 1.5 hrs.

The reaction was then concentrated and the residue dissolved in diethyl ether (1.25 l), ice water (500 g) was added and the mixture stirred for 1 hr. The organic was collected and the aqueous washed with further diethyl ether (2×500 ml). The combined organics were dried (Na₂SO₃) and concentrated to give methyl 3-ethyl-2-(formylamino)-2-pentenoate as a yellow oil, 77.8 g, 74.8%.

HPLC Rt=2.2 min (gradient 1); m/z [M+H]⁺=186

¹H NMR (CDCl₃): δ 1.04-1.15 (6H, m), δ 2.21 (1H, q, J=6 Hz), δ 2.31 (1H, q, J=6 Hz), δ 2.51 (1H, q, J=6 Hz), δ 2.6 (1H, q, J=6 Hz), δ 3.75 & δ 3.77 (3H, 2×s, rotomers), δ 6.68 & δ 6.78 (1H, broad), δ 7.96 (0.4H, m), δ 8.21 (0.6H, s).

Intermediate 2

### Methyl 3-Ethyl-N-formylnorvalinate

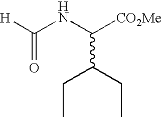

A solution of methyl 3-ethyl-2-(formylamino)-2-pentenoate (Intermediate 1) (47.12 g, 254 mmol) in acetic acid (250 ml) was hydrogenated at room temperature and pressure over 10% Pd/C (5.1 g) for 18 hours. The reaction was filtered through Celite®, washed with ethanol, and concentrated to give methyl 3-ethyl-N-formylnorvalinate as a yellow oil, 48.3 g, 102%.

HPLC Rt=2.32 min (gradient 1); m/z [M+H]⁺=188/189

¹H NMR (CDCl₃): δ 0.94 (6H, m), δ 1.3 (4H, m), δ 1.73 (1H, m), δ 3.76 (3H, m), δ 4.74 (0.13H, m), δ 4.87 (0.87H, m), δ 6.25 (0.87H, broad), δ 6.95 (0.13H, broad), δ 7.97 (0.13H, d, J=10 Hz), δ 8.25 (0.87H, s).

Intermediate 3

### Methyl 3-Ethylnorvalinate hydrochloride

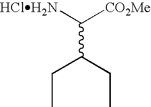

A solution of methyl 3-ethyl-N-formylnorvalinate (Intermediate 2) (31.1 g, 166 mmol) in methanol (490 ml) was cooled to 0° C. Acetyl chloride (44 ml) was added slowly with stirring. The reaction was then heated at 50° C. for 3 hrs. The reaction was then concentrated to provide methyl 3-ethylnorvalinate hydrochloride as a white solid, 30.78 g, 95%.

HPLC Rt=1.46 min (gradient 1); m/z [M+H]⁺=160/161

¹H NMR (CDCl₃): δ 0.99 (6H, q, J=4 Hz), δ 1.46 (2H, m), δ 1.75 (1H, m), δ 1.69 (1H, m), δ 1.95 (1H, m), δ 3.81 (3H, s), δ 4.11 (1H, m), δ 8.86 (3H, broad).

Intermediate 4

### (2R)-2,3-Dihydro-1H-inden-2-yl({[(phenylmethyl)oxy]carbonyl}amino)ethanoic acid

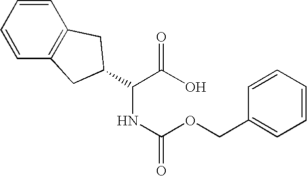

(2R)-Amino-(2,3-dihydro-1H-inden-2-yl)ethanoic acid (1.91 g, 10 mmol) was suspended in dioxane (10 ml) and water (10 ml). To this was added triethylamine (1.7 ml) and N-(benzyloxycarbonyloxy)-succinimde (2.54 g) and the reaction mixture was stirred rapidly at room temperature for 2 days. The reaction mixture was poured into water (50 ml) and extracted with chloroform (100 ml). The organic phase was washed with 1 N hydrochloric acid (50 ml) and water (50 ml). This was dried over magnesium sulphate and the solvent removed in vacuo to give the title compound (3.06 g, 94%).

HPLC Rt=3.35 min (gradient 2); LCMS m/z [MH⁺]=326

¹H NMR (CDCl₃) δ 7.40-7.29 (m, 5H), 7.21-7.11 (m, 4H), 5.28 (d, 1H, J=8.6 hZ), 5.11 (s, 2H), 4.57 (m, 1H), 3.14-2.79 (m, 5H).

Intermediate 5

### 2-[(3R,6R)-3-(2,3-Dihydro-1H-inden-2-yl)-6-(1-ethylpropyl)-2,5-dioxo-1-piperazinyl]-N-(2-hydroxyphenyl)-2-(6-methyl-3-pyridinyl)acetamide

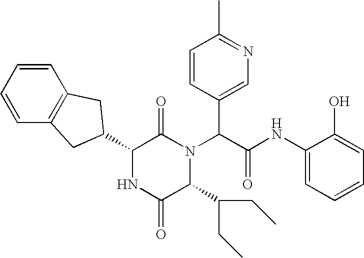

Methyl 3-ethylnorvalinate hydrochloride (Intermediate 3) (30 g) was dissolved in a mixture of trifluoroethanol (400 ml) and methanol (200 ml). Triethylamine (23.5 ml) and (2R)-[(benzyloxycarbonyl)amino]-(2,3-dihydro-1H-inden-2-yl)ethanoic acid (49.9 g) were added and the mixture stirred until the components were completely dissolved. 2-Benzyloxyphenylisocyanide (32.1 g) and 6-methyl-3-pyridinecarbaldehyde (J Org Chem, 53, 15, 1988, 3513) (18.6 g) were added and the reaction stirred for 18 hours at room temperature. The solvent was evaporated and the residue dissolved in ethanol (200 ml) and solvent evaporated a second time. The resulting residue was dissolved in ethanol (800 ml) and acetic acid (200 ml) and stirred under an atmosphere of hydrogen for 18 hours in the presence of palladium on carbon (16.3 g, 10% wt containing 50% water). The mixture was filtered through Celite®, evaporated and the residue dissolved in ethyl acetate (1 L) washed with water (2×400 ml), saturated sodium bicarbonate solution (1×400 ml) and dried over sodium sulfate. The crude product was purified by column chromatography (silica): Pre-elution of the trans-isomer with 75:25 ethyl acetate:cyclohexane was followed by elution with 100:0 to 80:20 ethyl actetate:ethanol to give the title compound as a brown solid (19.02 g, 22.8%).

HPLC (2 isomers): Rt=3.01 and 3.05 minutes (gradient 2); m/z [M+H]⁺=541.

Intermediate 6

### 2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N-(2-hydroxyphenyl)acetamide

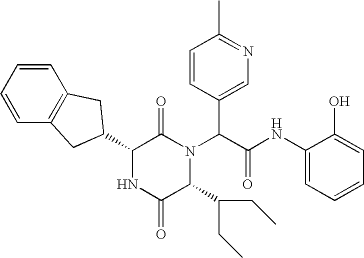

Similarly prepared from 2,6-dimethyl-3-pyridinecarbaldehyde (Aurora Feinchemie GmbH) using Intermediate 14.

HPLC (2 isomers): Rt=2.95 minutes, 3.01 minutes (gradient 2); m/z [M+H]⁺=555

Intermediate 7

### 2-[(3R,6R)-3-(2,3-Dihydro-1H-inden-2-yl)-6-(1-ethylpropyl)-2,5-dioxo-1-piperazinyl]-N-(2-hydroxyphenyl)-2-(1-methyl-1H-indazol-5-yl)acetamide

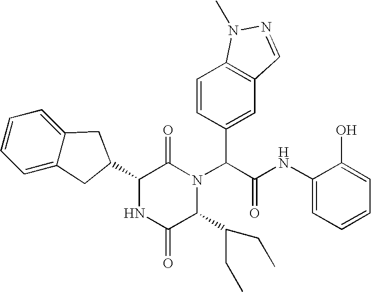

Similarly prepared from 1-methyl-1H-indazole-5-carbaldehyde (Intermediate 15) using Intermediate 14.

HPLC Rt=3.35 minutes (gradient 2); m/z [M+H]⁺=580

Intermediate 8

### 2-[(3R,6R)-3-(2,3-Dihydro-1H-inden-2-yl)-6-(1-ethylpropyl)-2,5-dioxo-1-piperazinyl]-N-(2-hydroxyphenyl)-2-(2-methyl-1,3-oxazol-4-yl)acetamide

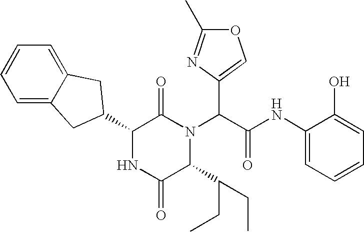

Similarly prepared from 2-methyl-1,3-oxazole-4-carbaldehyde (D L Boger, T T Curran, J. Org. Chem., 1992, 57 (8) 2235-2244) using intermediate 3.

HPLC Rt=3.32, 3.37 minutes (gradient 1); m/z [M+H]⁺=531.

Intermediate 9

### [(3R,6R)-3-(2,3-Dihydro-1H-inden-2-yl)-6-(1-ethylpropyl)-2,5-dioxo-1-piperazinyl]-(6-methyl-3-pyridinyl)acetic acid

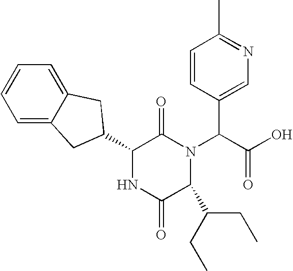

1,1′-Carbonyldiimidazole (590 mg, 1.6 equiv.) was added to a solution 2-[(3R,6R)-3-(2,3-dihydro-1H-inden-2-yl)-6-(1-ethylpropyl)-2,5-dioxo-1-piperazinyl]-N-(2-hydroxyphenyl)-2-(6-methyl-3-pyridinyl)acetamide (1.3 g) in dichloromethane (60 ml) and the solution stirred at room temperature for 16 hr. The mixture was evaporated and the residue dissolved in acetone (30 ml) and water (15 ml) added and the mixture stirred at room temperature for 7 hr. The solution was reduced in vacuo, dissolved in methanol (10 ml) and 4 equal aliquots of this solution loaded onto 4×10 g aminopropyl SPE cartridges. Each cartridge was washed with methanol (60 ml) and the product eluted with 10% acetic acid in methanol. Combination of the appropriate fractions, reduction in vacuo and azeotropic removal of the excess acetic acid with dioxan (2×10 ml) and dichloromethane (2×10 ml) gave [(3R,6R)-3-(2,3-dihydro-1H-inden-2-yl) -6-(1-ethylpropyl)-2,5-dioxo-1-piperazinyl](6-methyl-3-pyridinyl)acetic acid, 1.18 g (95%).

HPLC Rt=2.6 minutes (gradient 1); m/z [M+H]⁺=450

Intermediate 10

### [(3R,6R)-3-(2,3-Dihydro-1H-inden-2-yl)-6-(1-ethylpropyl)-2,5-dioxo-1-piperazinyl]-(2,6-dimethyl-3-pyridinyl)acetic acid

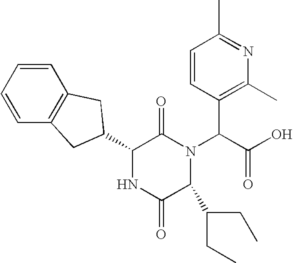

Similarly prepared from Intermediate 6.

HPLC Rt=2.50 minutes (gradient 2); m/z [M+H]⁺=464

Intermediate 11

### [(3R,6R)-3-(2,3-Dihydro-1H-inden-2-yl)-6-(1-ethylpropyl)-2,5-dioxo-1-piperazinyl]-(1-methyl-1H-indazol-5-yl)acetic acid

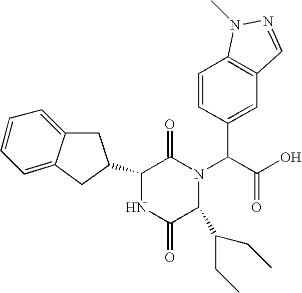

Similarly prepared from Intermediate 7.

HPLC (2 isomers): Rt=3.13 and 3.17 minutes (gradient 2); HPLC m/z [M+H]⁺=489

Intermediate 12

### [(3R,6R)-3-(2,3-Dihydro-1H-inden-2-yl)-6-(1-ethylpropyl)-2,5-dioxo-1-piperazinyl]-(2-methyl-1,3-oxazol-4-yl)acetic acid

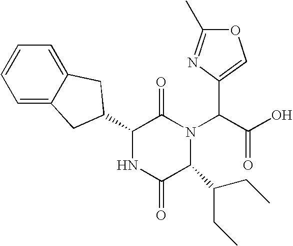

Similarly prepared from Intermediate 8.

HPLC (2 isomers): Rt=3.01 and 3.17 minutes (gradient 1); HPLC m/z [M+H]⁺=440

Intermediate 13

### Methyl 3-Ethyl-N-formyl-D-norvalinate

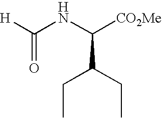

Intermediate 2 (a racemic mixture) (25 g) was purified on a Chiralpak AD column (2 in×20 cm, self packed, flow rate=75 ml/min) using 95:5 Heptane:EtOH as the eluent. Concentration yielded: Isomer 1: pale yellow solid, 8.67 g, Chiral HPLC Rt=6.28 mins and Isomer 2: yellow solid, 7.82 g, Chiral HPLC Rt=8.7 mins

Exact stereochemistry of the isomers was determined by obtaining optical rotations on the hydrochloride salts of the deprotected amino ester (see intermediate 14) according to the literature reference Collect. Czech. Chem. Commun, 31, 1966, 4563-4580. This determined that the title compound was Isomer 1 (HPLC Rt=2.37 min (gradient 2); HPLC m/z [M+H]⁺=188)

Intermediate 14

### Methyl 3-Ethyl-D-norvalinate hydrochloride

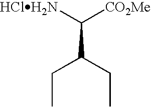

Deprotection of methyl 3-ethyl-N-formylnorvalinate (8.67 g) Isomer 1 (Intermediate 13) was carried out according to the procedure described for Intermediate 3 to give the corresponding hydrochloride salt of the amino ester (methyl 3-ethylnorvalinate hydrochloride, Isomer 1a). Isomer 1a was obtained as a white solid (8.42 g) and was confirmed as the R (D) isomer (title compound) according to the procedure detailed in Czech. Chem. Commun, 31, 1966, 4563-4580 (c=0.36 g/100 ml, lambda=589 nm, solvent=methanol). The title compound displayed an alpha D=−38°. ¹H NMR (DMSO): δ 8.50 (s, 3H), 3.97 (d, 1H), 3.75 (s, 3H), 1.71 (m, 1H), 1.45 (m, 1H), 1.33 (m, 1H), 1.18 (m, 1H), 0.89 (m, 6H)

Intermediate 15 (Method A)

### 1-Methyl-1H-indazole-5-carbaldehyde

A 2.0M solution of n-butyl magnesium chloride in tetrahydrofuran (3.05 ml) was added to toluene (20 ml) under nitrogen and cooled to −10° C. To this was added a 1.6M solution of n-butyl lithium in hexanes (7.63 ml) and after 1 hour the reaction mixture was cooled to −30° C. To this was added a solution of 5-bromo-1-methyl-1H-indazole¹ (2.35 g) in tetrahydrofuran (10 ml) and the reaction mixture was warmed to −10° C. After 1 hour dimethylformamide (5 ml) was added and the reaction mixture was stirred at −10° C. for 1 hour. The reaction was quenched using 2N hydrochloric acid (20 ml) and the reaction allowed to warm to room temperature. After 30 minutes the reaction mixture was basified with saturated aqueous sodium bicarbonate solution and then extracted using ethyl acetate (2×80 ml). The organic phase was washed with sodium bicarbonate solution (2×100 ml) and then 10% lithium chloride in water (2×100 ml) and then brine. The organic phase was dried over anhydrous magnesium sulphate and evaporated in vacuo. The residue was applied to a silica Redisep® cartridge (120 g) and eluted with 10-30% ethyl acetate in cyclohexane. The required fractions were combined and evaporated in vacuo to give 1-methyl-1H-indazole-5-carbaldehyde (1.43 g, 80%) as a white solid.

HPLC Rt=2.2 minutes (gradient 1); m/z [M+H]⁺=161 (gradient 1)

Intermediate 15 (Method B)

### 1-Methyl-1H-indazole-5-carbaldehyde

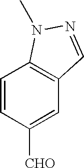

To a solution of 1-methyl-1H-indazole-5-carbonitrile² (7 g) in anhydrous toluene (300 ml) under nitrogen at −70° C. was added a 1.5M solution of DIBAL in toluene (59.4 ml) drop wise over approx 20 minutes. The reaction mixture was allowed to warm to −60° C. and stirred at that temperature for 4 hours, the cooling bath removed and then quenched by drop wise addition of acetic acid (30 ml) (care evolution of gas). Water (240 ml) was added and mixture vigorously stirred for 30 minutes and then extracted with ethyl acetate (200 ml). The organic phase was washed with water (100 ml) and then brine (100 ml) dried over anhydrous magnesium sulphate, filtered and concentrated in vacuo to give 1-methyl-1H-indazole-5-carbaldehyde (6.8 g, 95%) as a pale yellow solid, consistent in all respects with that obtained from 5-bromo-1-methyl-1H-indazole obtained above.

### EXAMPLE 1

### (2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide

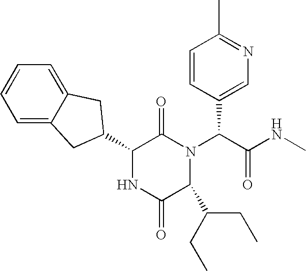

To a solution of [(3R,6R)-3-(2,3-dihydro-1H-inden-2-yl)-6-(1-ethylpropyl)-2,5-dioxo-1-piperazinyl](6-methyl-3-pyridinyl)acetic acid (Intermediate 9) (800 mg) in dichloromethane (9 ml) was added N,N-diisopropylethylamine (0.34 ml) and TBTU (630 g). The mixture was stirred at room temperature for 30 minutes whereupon methylamine (2.7 ml, 2M tetrahydrofuran) was added and the mixture stirred for 18 hours. The solvent was removed in vacuo and the mixture purified by column chromatography (silica) eluting with 1-10% ethanol in ethyl acetate and preparative HPLC to give (2R)-2-[(3R,6R)-3-(2,3-dihydro-1H-inden-2-yl)-6-(1-ethylpropyl)-2,5-dioxo-1-piperazinyl]-N-methyl-2-(6-methyl-3-pyridinyl)ethanamide, (150 mg)

HPLC Rt=2.6 minutes (gradient 2); m/z [M+H]⁺=463

¹H NMR (CDCl₃) δ 8.50 (d, 1H), 7.75 (dd, 1H), 7.32 (d, 1H), 7.15-7.3 (m, 5H), 6.32 (br.q, 1H), 4.87 (s, 1H), 4.14 (d, 1H), 4.06 (dd, 1H), 3.05-3.20 (m, 3H), 2.95 (pentet, 1H), 2.87 (d, 3H), 2.81 (dd, 1H), 2.61 (s, 3H), 1.83 (m, 1H), 1.72 (m, 3H), 1.27 (m, 1H), 1.01 (m, 6H).

### EXAMPLE 2

### (2R)-2-[(3R,6R)-3-(2,3-Dihydro-1H-inden-2-yl)-6-(1-ethylpropyl)-2,5-dioxo-1-piperazinyl]-N,N-dimethyl-2-(6-methyl-3-pyridinyl)ethanamide

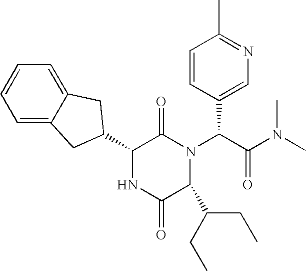

To a solution of 2-[(3R,6R)-3-(2,3-dihydro-1H-inden-2-yl)-6-(1-ethylpropyl)-2,5-dioxo-1-piperazinyl]-N-(2-hydroxyphenyl)-2-(6-methyl-3-pyridinyl)acetamide (220 mg) in dichloromethane (2 ml) was added 1,1′-carbonyldiimidazole (110 mg) and ca 4 pellets of 3 Å activated molecular sieves. The mixture was stirred for 18 hours, cooled to 0° C. and dimethylamine (1.2 ml, 2M THF) added. Upon stirring at room temperature for a further 4 hours the mixture was reduced in vacuo and purified by column chromatography (silica) eluting with 1-10% ethanol in ethyl acetate and preparative HPLC (MDAP) to give (2R)-2-[(3R,6R)-3-(2,3-dihydro-1H-inden-2-yl)-6-(1-ethylpropyl)-2,5-dioxo-1-piperazinyl]-N,N-dimethyl-2-(6-methyl-3-pyridinyl)ethanamide, 49 mg (26%).

HPLC Rt=2.9 minutes (gradient 1); m/z [M+H]⁺=477

¹H NMR (CDCl₃) δ 8.57 (d, 1H), 7.79 (dd, 1H), 7.43 (d, 1H), 7.1-7.3 (m, 6H), 6.21 (s, 1H), 4.18 (d, 1H), 4.11 (dd, 1H), 3.10-3.20 (m, 3H), 2.95 (d, 3H), 2.75-2.95 (m, 2H), 2.62 (s, 3H), 1.42 (m, 1H), 1.23 (sextet, 1H), 1.10 (m, 2H), 0.92 (m, 1H), 0.73 (t, 3H), 0.44 (t, 3H).

### EXAMPLE 3

### (2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N-methylethanamide

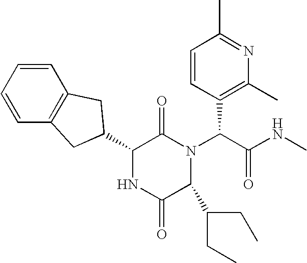

Similarly prepared from Intermediate 10 and methylamine using CDI as a coupling agent according to the method described in Example 4.

HPLC Rt=2.59 minutes (gradient 2); m/z [M+H]⁺=477;

¹H NMR (methanol d-4) δ7.65 (d, 1H), 7.23 (d, 1H), 7.21-7.09 (m, 4H), 6.10 (s, 1H), 4.17 (d, 1H), 4.05 (d, 1H), 3.12-2.77 (m, 5H), 2.72 (s, 3H), 2.58 (s, 3H), 2.52 (s, 3H), 1.60-1.49 and 1.23-0.92 (3m, 4H), 0.88-0.80 (m, 1H), 0.75 (t, 3H), 0.53 (t, 3H).

### EXAMPLE 4

### (2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N,N-dimethylethanamide

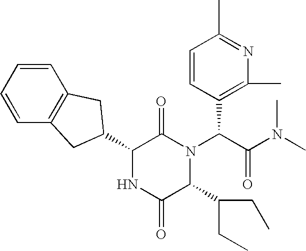

Intermediate 10 (700 mg, 1.510 mmol) and 1,1′-carbonyldiimidazole (CDI) (400 mg, excess) were stirred at room temperature in dry dichloromethane (10 mL) for 48 hours then a solution of dimethylamine (2.0M) in tetrahydrofuran (15 mL, 30 mmol) was added. After a further 2 hours LCMS showed no starting acid remaining. The solvents plus excess of dimethylamine were removed under reduced pressure and the residue was taken up in dichloromethane (50 mL) and the solution was washed with saturated aqueous sodium hydrogen carbonate (10 mL). The organic phase was separated (hydrophobic frit) and evaporated under reduced pressure, and the residue was purified on a 20 g silica SPE cartridge eluted with 2%, 3% and 5% solutions of isopropanol in dichloromethane to give the title compound as a pale yellow solid:

HPLC Rt=2.85 minutes (gradient 2); m/z [M+H]⁺=491;

¹H NMR (methanol d-4) δ67.54 (d, 1H), 7.26 (d, 1H), 7.21-7.09 (m, 4H), 6.63 (s, 1H), 6.48 (s, 1H), 4.21 (d, 1H), 4.02 (d, 1H), 3.14-2.74 (m, 11H), 2.60 and 2.54 (2s, 6H), 1.60-1.49 and 1.23-0.92 (3m, 4H), 0.71 (t, 3H), 0.60-0.50 (m, 1H), 0.45 (t, 3H).

### EXAMPLE 5

### (3R,6R)-3-(2,3-Dihydro-1H-inden-2-yl)-1-[(1R)-1-(2,6-dimethyl-3-pyridinyl)-2-(4-morpholinyl)-2-oxoethyl]-6-(1-ethylpropyl)-2,5-piperazinedione

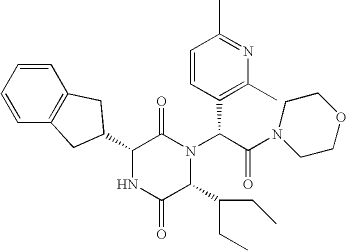

Similarly prepared from Intermediate 10 and morpholine using CDI as a coupling agent.

HPLC Rt=2.85 minutes (gradient 2); m/z [M+H]⁺=533;

¹H NMR (methanol d-4) δ 7.59 (d, 1H), 7.23 (d, 1H), 7.21-7.09 (m, 4H), 6.64 (s, 1H), 4.18 (d, 1H), 4.03 (d, 1H), 3.68-3.51, 3.34-3.20, 3.14-2.78 (3m, 13H), 2.60 (s, 3H), 2.55 (s, 3H), 1.60-1.49 and 1.23-0.91 (3m, 4H), 0.71 (t, 3H), 0.60-0.50 (m, 1H), 0.46 (t, 3H).

### EXAMPLE 6

### (2R)-2-[(3R,6R)-3-(2,3-Dihydro-1H-inden-2-yl)-6-(1-ethylpropyl)-2,5-dioxo-1-piperazinyl]-N-methyl-2-(1-methyl-1H-indazol-5-yl)acetamide

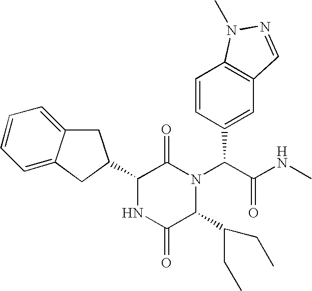

Similarly prepared from Intermediate 11 and methylamine using CDI as a coupling agent.

HPLC Rt=2.96 minutes (gradient 2); m/z [M+H]⁺=502

¹H NMR (CDCl₃) δ 8.00 (d, 1H), 7.80 (s, 1H), 7.49 (dd, 1H), 7.42 (d, 1H), 7.26-7.14 (m, 4H), 6.83 (br. d, 1H), 6.09 (q, 1H), 4.91 (s, 1H), 4.15 (d, 1H), 4.12-4.06 (m, 4H), 3.25-2.92 (m, 5H), 2.85 (d, 3H), 2.81-2.73 (dd, 1H), 1.85-1.69 (m, 2H), 1.64 (m, 1H), 1.31-1.19 (m, 1H), 0.96 (t, 3H), 0.91 (t, 3H).

### EXAMPLE 7

### (2R)-2-[(3R,6R)-3-(2,3-Dihydro-1H-inden-2-yl)-6-(1-ethylpropyl)-2,5-dioxo-1-piperazinyl]-N,N-dimethyl-2-(1-methyl-1H-indazol-5-yl)ethanamide

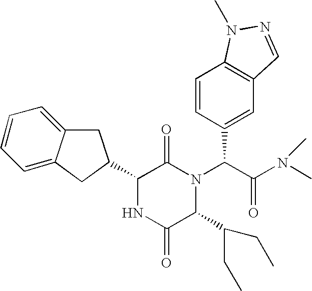

Similarly prepared from Intermediate 11 and dimethylamine dimethylamine using CDI as a coupling agent.

HPLC Rt=3.08 minutes (gradient 2); m/z [M+H]⁺=516

¹H NMR (CDCl₃) δ8.02 (s, 1H), 7.82 (s, 1H), 7.50 (dd, 1H), 7.46 (d, 1H), 7.27-7.13 (m, 4H), 6.43 (m, 2H), 4.40 (d, 1H), 4.16 (dd, 1H), 4.12 (s, 3H), 3.24-3.10 (m, 3H), 2.97 (s, 3H), 2.97-2.89 (m, 1H), 2.89 (s, 3H), 2.75 (dd, 1H) 1.43 (m, 1H), 1.19-0.87 (m, 3H), 0.75 (m, 1H), 0.62 (t, 3H), 0.21 (t, 3H).

### EXAMPLE 8

### (3R,6R)-3-(2,3-Dihydro-1H-inden-2-yl)-6-(1-ethylpropyl)-1-[(1R)-1-(1-methyl-1H-indazol-5-yl)-2-(4-morpholinyl)-2-oxoethyl]-2,5-piperazinedione

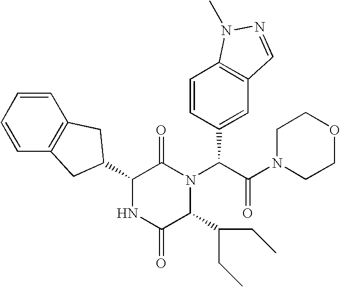

Similarly prepared from Intermediate 11 and morpholine morpholine using CDI as a coupling agent.

HPLC Rt=3.04 minutes (gradient 2); m/z [M+H]⁺=558

¹H NMR (CDCl₃) δ 8.03 (s, 1H), 7.80 (s, 1H), 7.48 (d, 1H), 7.44 (dd, 1H), 7.27-7.12 (m, 4H), 6.49 (s, 1H), 6.37 (d, 1H), 4.37 (d, 1H), 4.15 (dd, 1H), 4.13 (s, 3H), 3.74-3.40 (m, 6H), 3.22-3.10 (m, 4H), 3.06 (m, 1H), 2.94 (m, 1H), 2.76 (dd, 1H), 1.43 (m, 1H), 1.18-1.00 (m, 2H), 0.89 (m, 1H), 0.73 (m, 1H), 0.61 (t, 3H), 0.25 (t, 3H).

### EXAMPLE 9

### (3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione

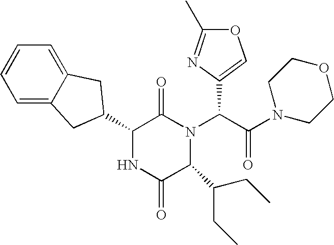

Prepared from Intermediate 12 and morpholine:

HPLC Rt=2.91 minutes (gradient 1); m/z [M+H]⁺=509

1H NMR (CDCl₃) δ 7.71 (s, 1H), 7.16-7.26 (m, 4H), 6.54 (broad d, 1H), 6.26 (s, 1H), 4.38 (d, 1H), 4.09 (dd, 1H), 3.70-3.64 (m, 5H), 3.49 (m, 1H), 3.46 (m, 1H), 3.45 (m, 1H), 3.17-3.12 (m, 3H), 2.95 (m, 1H), 2.75 (m, 1H), 2.49 (s, 3H), 1.57-1.5 (partially obscured by H₂0, m, 1H), 1.42-1.38 (m, 1H), 1.33-1.23 (m, 1H), 1.13 (m, 1H), 0.95 (m, 1H), 0.77 (t, 3H), 0.71 (t, 3H).

Biological Activity

Examples 1 to 9 of the present invention were tested in all of the assays described below. Results from Assay 1 for each of the compounds are shown in Table 1 below. The table also includes two compounds X and Y for comparison.

Assay 1

Determination of Antagonist Affinity at Human Oxytocin-1 Receptors Using FLIPR

Cell Culture

Adherent Chinese Hamster Ovary (CHO) cells, stably expressing the recombinant human Oxytocin-1 (hOT) receptor, were maintained in culture in DMEM:F12 medium (Sigma, cat no D6421), supplemented with 10% heat inactivated foetal calf serum (Gibco/Invitrogen, cat. no. 01000-147), 2 mM L-glutamine (Gibco/Invitrogen, cat. no. 25030-024) and 0.2 mg/ml G418 (Gibco/Invitrogen, cat no. 10131-027). Cells were grown as monolayers under 95%:5% air:CO₂ at 37° C. and passaged every 3-4 days using TrypLE™ Express (Gibco/Invitrogen, cat no. 12604-013).

Measurement of [Ca²⁺]ᵢ Using the FLIPR™

CHO-hOT cells were seeded into black walled clear-base 384-well plates (Nunc) at a density of 10,000 cells per well in culture medium as described above and maintained overnight (95%:5% air:CO₂ at 37° C.). After removal of culture medium, cells were incubated for 1 h at 37° C. in Tyrode's medium (NaCl, 145 mM; KCl, 2.5 mM; HEPES, 10 mM; Glucose, 10 mM; MgCl₂, 1.2 mM; CaCl₂, 1.5 mM) containing probenacid (0.7 mg/ml), the cytoplasmic calcium indicator, Fluo-4 (4 uM; Teflabs, USA) and the quenching agent Brilliant Black (250 uM; Molecular Devices, UK). Cells were then incubated for an additional 30 min at 37° C. with either buffer alone or buffer containing OT antagonist, before being placed into a FLIPR™ (Molecular Devices, UK) to monitor cell fluorescence (λₑₓ=488 nm, λ_{EM}=540 nm) before and after the addition of a submaximal concentration of oxytocin (EC80).

Data Analysis

Functional responses using FLIPR were analysed using Activity Base Version 5.0.10. Data are reported as mean values, wherein number of times tested (n) is greater than or equal to 3.

Assay 2

Oxytocin Binding Assay

Preparations

Membranes were prepared from CHO cells expressing human recombinant oxytocin receptors. The membrane preparation was frozen in aliquots at −70° C. until used.

Binding Assay Protocol

Membranes (˜50 ug) were incubated in 200 ul of assay buffer (50 mM Tris, 10 mM MgCl₂, and 0.1% bovine serum albumin, pH 7.5) containing ˜2.4 nM of [3H]-oxytocin in the absence (total binding) or presence (non-specific binding) of 1 uM unlabeled oxytocin and increasing concentrations of the compounds in Examples 1 to 9 or comparator compounds. Incubations were performed at room temperature for 60 minutes. The reactions were stopped with 3 ml of ice cold buffer and filtered through Whatman GF/C filter paper presoaked in 0.3% polyethylenimine. The filters were washed 4 times with 3 ml buffer using a Brandel cell harvester. The filters were counted in 3 ml Ready Safe scintillation fluid (Beckman).

Specific binding represented approximately 90% of total binding.

Data Analysis

IC₅₀ values were determined from competition binding experiments using non-linear regression analysis (GraphPad) and converted to Ki using the method of Cheng and Prusoff, 1974. Data are reported as mean values.

Assay 3

Determination of In vitro Intrinsic Clearance in Microsomes

NADP regeneration buffer for use in incubations was prepared fresh on the assay day. It contained 7.8 mg glucose-6-phosphate (mono-sodium salt), 1.7 mg NADP and 6 Units glucose-6-phosphate dehydrogenase per 1 mL of 2% sodium bicarbonate. Microsomes (human, female; cynomolgus monkey, female; dog, female; rat, female) were prepared in pH 7.4 phosphate buffer and contained 0.625 mg protein/mL. Unless stated, all subsequent steps were performed by a Tecan Genesis 150/8 RSP. A 1.25 mM stock solution of the compounds was prepared in acetonitrile/water (1:1). 25 ul of the 1.25 mM stock solution was added to 600 ul of acetonitrile/water (1:1) to give a 50 uM solution. For each species, the 50 uM solutions (10 uL) were added to microsomes (790 uL) in a microplate (Porvair, 96 deepwell, square).

400 uL of the microsomal solution containing the compound was transferred to a microplate (Porvair, 96 deepwell, round) and was pre-warmed at 37° C. for five minutes prior to initiation of incubations. All incubations were initiated by addition of 100 uL of NADP regeneration system to the pre-warmed microsomes. The mixtures were incubated at 37° C. in a Techne heating block. Following 0, 3, 6, 12 and 30 minutes incubation, 20 uL aliquots were taken and added to 100 uL of acetonitrile containing internal standard.

For determination of the rate of metabolism, incubations were performed at a compound concentration of 0.5 uM and a protein concentration of 0.5 mg/mL. The concentration of solvent in the incubation was 0.5%.

Test compound concentrations were determined by LC/MS/MS; results were reported as analyte:internal standard peak area ratios.

The rate of disappearance was calculated by fitting a single exponential decay to the concentration-time curve using Excel and intrinsic clearance was calculated using the following formula:

$$Cli = \frac{[ rate ( 1 / min ) * 52.5 mg protein / g liver ]}{0.5 mg protein / mL}$$

Results

Examples 1 to 9 of the present invention and also two comparator compounds [Comparator compound X=(2R)-2-[(3R,6R)-3-(2,3-dihydro-1H-inden-2-yl)-6-isobutyl-2,5-dioxopiperazin-1-yl]-N,N-dimethyl-2-(6-methylpyridin-3-yl)ethanamide (Example 209 in WO 03/053443), and Comparator compound Y=(3R,6R)-3-(2,3-dihydro-1H-inden-2-yl)-1-[(1R)-1-(2-methyl-1,3-oxazol-4-yl)-2-(4-morpholinyl)-2-oxoethyl]-6-(2-methylpropyl)-2,5-piperazinedione (Example 180 in WO 03/053443)] were tested in the above assays, except that example 7 was not tested in Assay 3 (Determination of In-Vitro Intrinsic Clearance in Microsomes).

The results of testing examples 1 to 9 and Comparator compounds X and Y in Assay 1 (antagonist affinity at human Oxytocin-1 receptors using FLIPR) are shown in Table 1. Examples 1 to 9 showed a surprisingly improved antagonist affinity as compared with that of Comparator compound X and Comparator compound Y.

Examples 1 to 6, 8 and 9 showed a comparable in vitro intrinsic clearance in microsomes (Assay 3) to that of Comparator compounds X and Y.

TABLE 1

hOT FLIPR

| | fpKi | |
|---|---|---|
| Assay 1 | mean | n |
| Comparator X | ++ | 5 |
| Comparator Y | + | 5 |
| Example 1 | +++ | 22 |
| Example 2 | ++++ | 13 |
| Example 3 | ++++ | 3 |
| Example 4 | ++++ | 3 |
| Example 5 | +++++ | 3 |
| Example 6 | +++++ | 3 |
| Example 7 | +++++ | 3 |
| Example 8 | +++++ | 3 |
| Example 9 | ++++ | 4 |

Key to Table 1
n = number of times tested (mean fpKi value shown)
+ corresponds to fpKi of 7.0-7.5
++ corresponds to fpKi of 7.6-8.1
+++ corresponds to fpKi of 8.2-8.7
++++ corresponds to fpKi of 8.8-9.3
+++++ corresponds to fpKi of 9.4 or greater

What is claimed is:

1. A compound of formula (I) 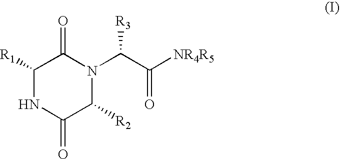
wherein R₁ is 2-indanyl, R₂ is 1-ethylpropyl, R₃ is a heterocyclic group optionally substituted by one or more C₁₋₆ alkyl groups, R₄ represents methyl and R₅ represents hydrogen or methyl and pharmaceutically acceptable derivatives thereof.

2. A salt or solvate of formula (I), according to claim 1, 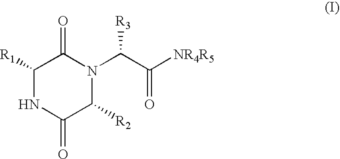
wherein R₁ is 2-indanyl, R₂ is 1-ethylpropyl, R₃ is a heterocyclic group optionally substituted by one or more C₁₋₆ alkyl groups, R₄ represents methyl and R₅ represents hydrogen or methyl.

3. A compound of formula (IA), according to claim 1, 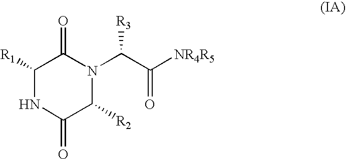
wherein R₁ is a 2-indanyl, R₂ is 1-ethylpropyl, R₃ is 4-methyl-3-pyridinyl, R₄ represents methyl and R₅ represents hydrogen or methyl and pharmaceutically acceptable derivatives thereof.

4. A compound according to claim 1 wherein R₃ is indazolyl, pyridinyl or oxazolyl, any of which may be optionally substituted by one or more C₁₋₆ alkyl groups.

5. A compound according to claim 1 wherein R₃ is indazolyl optionally substituted by one or more C₁₋₆ alkyl groups.

6. A compound according to claim 1 wherein R₃ is pyridinyl optionally substituted by one or more C₁₋₆ alkyl groups.

7. A compound according to claim 1 wherein R₃ is oxazolyl optionally substituted by one or more C₁₋₆ alkyl groups.

8. A compound according to claim 1 selected from:
(2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide;
(2R)-2-[(3R,6R)-3-(2,3-Dihydro-1H-inden-2-yl)-6-(1-ethylpropyl)-2,5-dioxo-1-piperazinyl]-N,N-dimethyl-2-(6-methyl-3-pyridinyl)ethanamide;
(2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N-methylethanamide;
(2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N,N-dimethylethanamide;
(3R,6R)-3-(2,3-Dihydro-1H-inden-2-yl)-1-[(1R)-1-(2,6-dimethyl-3-pyridinyl)-2-(4-morpholinyl)-2-oxoethyl]-6-(1-ethylpropyl)-2,5-piperazinedione;
(2R)-2-[(3R,6R)-3-(2,3-Dihydro-1H-inden-2-yl)-6-(1-ethylpropyl)-2,5-dioxo-1-piperazinyl]-N-methyl-2-(1-methyl-1H-indazol-5-yl)acetamide;
(2R)-2-[(3R,6R)-3-(2,3-Dihydro-1H-inden-2-yl)-6-(1-ethylpropyl)-2,5-dioxo-1-piperazinyl]-N,N-dimethyl-2-(1-methyl-1H-indazol-5-yl)ethanamide;
(3R,6R)-3-(2,3-Dihydro-1H-inden-2-yl)-6-(1-ethylpropyl)-1-[(1R)-1-(1-methyl-1H-indazol-5-yl)-2-(4-morpholinyl)-2-oxoethyl]-2,5-piperazinedione;
(3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione;
and pharmaceutically acceptable derivatives thereof.

9. A compound according to claim 1 selected from:
(2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide;
(2R)-2-[(3R,6R)-3-(2,3-Dihydro-1H-inden-2-yl)-6-(1-ethylpropyl)-2,5-dioxo-1-piperazinyl]-2-(2,6-dimethyl-3-pyridinyl)-N-methylethanamide;
(2R)-2-[(3R,6R)-3-(2,3-Dihydro-1H-inden-2-yl)-6-(1-ethylpropyl)-2,5-dioxo-1-piperazinyl]-N-methyl-2-(1-methyl-1H-indazol-5-yl)acetamide;
(3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione;
and pharmaceutically acceptable derivatives thereof.

10. A compound according to claim 1 selected from:
(2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide;
(3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione;
and pharmaceutically acceptable derivatives thereof.

11. A compound according to claim 1 selected from:
(2R)-2-[(3R,6R)-3-(2,3-Dihydro-1H-inden-2-yl)-6-(1-ethylpropyl)-2,5-dioxo-1-piperazinyl]-N-methyl-2-(6-methyl-3-pyridinyl)ethanamide;
and pharmaceutically acceptable derivatives thereof.

12. A compound according to claim 1 selected from:
(3R,6R)-3-(2,3-Dihydro-1H-inden-2-yl)-6-(1-ethylpropyl)-1-[(1R)-1-(2-methyl-1,3-oxazol-4-yl)-2-(4-morpholinyl)-2-oxoethyl]-2,5-piperazinedione;
and pharmaceutically acceptable derivatives thereof.

13. A pharmaceutical composition comprising a compound, according to claim 1, and a pharmaceutically acceptable carrier.

14-16. (canceled)

17. A method of treating or preventing diseases or conditions mediated through the action of oxytocin which comprises administering to a mammal in need thereof of an effective amount of at least one chemical entity according to claim 1.

18. A method according to claim 17 wherein the disease or condition is selected from pre-term labour, dysmenorrhea, endometriosis and benign prostatic hyperplasia.

19. A process for the preparation of compounds of formula (I) or of formula (IA) as claimed in claim 1 which comprises:
(a) reacting a compound of formula (II) 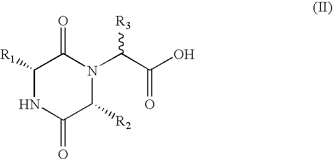
wherein R₁, R₂ and R₃ have the meanings defined in claim 1 and the chirality at R₃ is either R or S or a mixture thereof, or an activated derivative thereof, with the amine HNR₄R₅ wherein R₄ and R₅ have the meaning defined in claim 1 or under standard conditions for preparing amides from a carboxylic acid or an activated derivative thereof and an amine, or
(b) reacting a compound of formula (III) 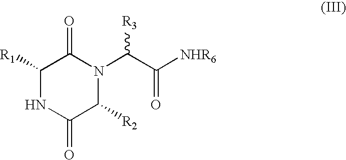
wherein R₁, R₂ and R₃ have the meanings defined in claim 1, and R₆ is 2-hydroxyphenyl, with 1,1′-carbonyldiimidazole or 1,1′-thiocarbonyldiimidazole in a suitable solvent and subsequent reaction of the product thus formed with amine HNR₄R₅ wherein R₄ and R₅ have the meanings defined in claim 1.
